(19) United States
(12) Patent Application Publication
(10) Pub. No.: US 2009/0204077 A1
(43) Pub. Date: Aug. 13, 2009

(54) Moulded Connection Between Cannula and Delivery Part

(76) Inventors: Soren B. Hasted, Kobenhaven O (DK); Elo L. Hordum, Horsholm (DK); Steffen Gyrn, Ringsted (DK)

Correspondence Address: BRINKS HOFER GILSON & LIONE, P.O. BOX 10395, CHICAGO, IL, 60610, US

(21) Appl. No.: 12/187,971

(22) Filed: Aug. 7, 2008

Related U.S. Application Data
(60) Provisional application No. 61/028,262, filed on Feb. 13, 2008.

(30) Foreign Application Priority Data
Feb. 13, 2008 (DK) PA 2008 00203

(51) Int. Cl. A61M 5/34

(52) U.S. Cl. 604/240

(57) ABSTRACT

A fluid connection for use between a reservoir and a cannula part of a medical device is provided. The fluid connection includes a tube of rigid material. The tube has a first end operably connected to the reservoir and a second end operably connected to the cannula part. The tube is operably attached to a surface plate of the medical device.

### RELATED APPLICATIONS

This application claims the benefit of U.S. Provisional Application No. 61/028,262, filed Feb. 13, 2008, and Danish Patent Application No. PA 2008 00203, filed Feb. 13, 2008. These references are incorporated by reference herein in their entirety.

### TECHNICAL FIELD

One aspect of the invention relates to a device for continuous administration of a therapeutically working substance, such as insulin, comprising a base part to which an injection part and a delivery part can be fastened. A delivery part normally comprises a reservoir and e.g. a pump, and the injection part comprises a body with a through-going opening, and at least one cannula having a proximal end protruding from the lower side of the body. One aspect of the invention comprises a fluid connection formed between a reservoir and a cannula part which fluid connection normally is attached unreleasably to the base plate and has the form of a tube made of a rigid material.

### BACKGROUND

WO 2007/071258 describes a medical device for delivering fluid comprising an injection part and a fluid delivery part where the fluid delivery part and the injection part can be separated and rejoined. The fluid delivery part comprises a reservoir, means for transport of liquid e.g. in form of a pump and a housing in which the active units of the delivery part is placed. The injection part comprises: a base plate, a cannula part comprising a body with a through going opening provided with a cannula extending past the proximal side of the base plate and means for fixation of the base plate to the skin of the user e.g. in the form of a mounting pad. The delivery part and the injection part is assembled through a connector comprising a fluid path leading fluid from the reservoir to the through-going opening in the cannula part which fluid path comprises means for blocking access to the injection part when the connector is disconnected from the delivery part and/or the injection part. FIG. 20-24 in this document illustrates an embodiment where the connector is constructed of a molded body fastened unreleasably to a base plate and provided with an interior compartment to which access is protected by a septum. The septum can be penetrated by a connector needle belonging to the delivery part when the delivery part is fastened to the base plate. From the lower part of the interior compartment of the connector, an opening 5a allows fluid to enter into a flexible tube and pass onto the cannula part 9. The flexible tube is connected to the first part of the injection part and when the second part 1b of the injection part is positioned in the first part 1a a fluid path is created from the flexible tube 5 to the cannula 9.

The embodiments illustrated in this document are quite complex and not easy to manufacture.

### SUMMARY OF THE INVENTION

An object of the invention is to provide a fluid connection between a reservoir and a cannula part which fluid connection is attached to a surface plate and has the form of a tube made of a rigid material. That the fluid connection is placed between the reservoir and the cannula part means that the fluid connection does not form part of either the reservoir or the cannula part e.g. the tube does not comprise the actual cannula which penetrates the patients skin. The tube can be releasably or unreleasably attached to the surface plate.

According to one embodiment the fluid connection is fastened to the surface plate by a holding part. The holding part 61 can either be made of one piece e.g. by moulding or combined from several pieces e.g. individual moulded pieces which after the individual manufacturing are assembled to the holding part 61.

According to one embodiment the tube is made of metal or plastic, e.g. the tube can comprise a hollow needle made e.g. of steel.

According to one embodiment the tube has a diameter or maximum cross-section smaller than 1 mm.

According to one embodiment the tube has at least one pointy end protruding from the holding part. That the end is “pointy” means that it has a sharp edge and can penetrate e.g. a protective membrane. This embodiment of the tube can be provided with a blunt end. That the end is “blunt” means that it does not have a sharp edge. Further according to this embodiment the pointy end of the tube can form a connector needle being the inlet to a connector part and when pushing a reservoir towards the inlet the connector needle penetrates a membrane completely covering a first opening of the connector part.

According to one embodiment the tube consist of a single piece of material i.e. it is not assembled by several pieces but is e.g. moulded or made by extrusion.

According to one embodiment the tube is bent at an angle >0 degrees in at least one position. According to this embodiment the tube can be bent in an angle >0 degrees in at least two positions. Normally the angle will be larger than 45 degrees and according to one embodiment the angle is around 90 degrees. Further the angles of the two positions will normally be identical.

According to one embodiment the tube is stationary in relation to the surface plate after the tube has been attached to the surface plate.

According to one embodiment the contact surface of the surface plate has the same size as a credit card i.e. it covers a similar area and the length of the fluid path provided by the connection is max. 3 cm.

### BRIEF DESCRIPTION OF THE DRAWING

Embodiments of the invention will now be described with reference to the figures in which:

FIG. 1 shows a first embodiment of a base part seen from above where a delivery part is connected through a membrane at a first opening and a cannula unit can be connected at a second opening.

FIG. 2A shows the embodiment of FIG. 1 joined with a delivery part and an inserter.

FIG. 2B shows the same embodiment as FIG. 2A seen from the end where the inserter is positioned.

FIG. 3 shows the embodiment of FIG. 1 joined with a delivery part and the cannula part in a pre-inserted position.

FIG. 4 shows the embodiment of FIG. 3 seen from the end where the connection part is placed.

FIG. 5A shows an embodiment of the base part provided with a surface plate which plate provides an outer cover for the connection part.

FIG. 5B shows the same embodiment as FIG. 5A of the base part where the outer cover has been removed.

FIG. 6 shows an embodiment of the base part where the outer cover has been removed and the membrane covering the entrance from the reservoir also has been removed.

FIG. 7 shows a second embodiment of the invention where the base part is provided with two longitudinal guiding means.

FIG. 8 shows a first embodiment of a cannula part.

FIG. 9 shows a second embodiment of a cannula.

FIGS. 10A, B and C show one embodiment of a connection part according to the invention including the internal parts.

FIG. 11A-E shows several embodiments of sealings in the form of a bubble shaped membranes.

FIGS. 12A and 12B show a second embodiment of a connection according to the invention including the internal parts.

FIGS. 13A and 13B show a third embodiment of a connection according to the invention including the internal parts.

### DETAILED DESCRIPTION

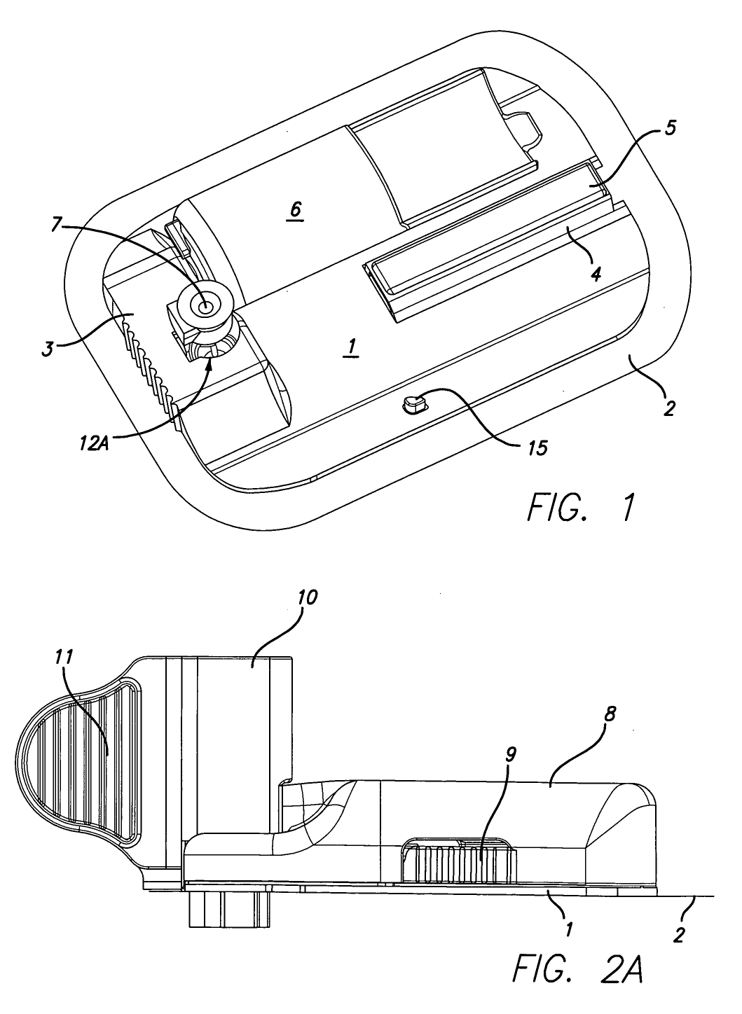

The FIGS. 1-6 show a first embodiment of a base part according to the invention. This embodiment comprises a surface plate 1 attached to a contact surface. The surface plate 1 is in this embodiment constructed of a molded plastic material and the contact surface is the proximal side of a mounting pad 2 which mounting pad 2 is unreleasably fastened to the surface plate 1 during manufacturing of the device.

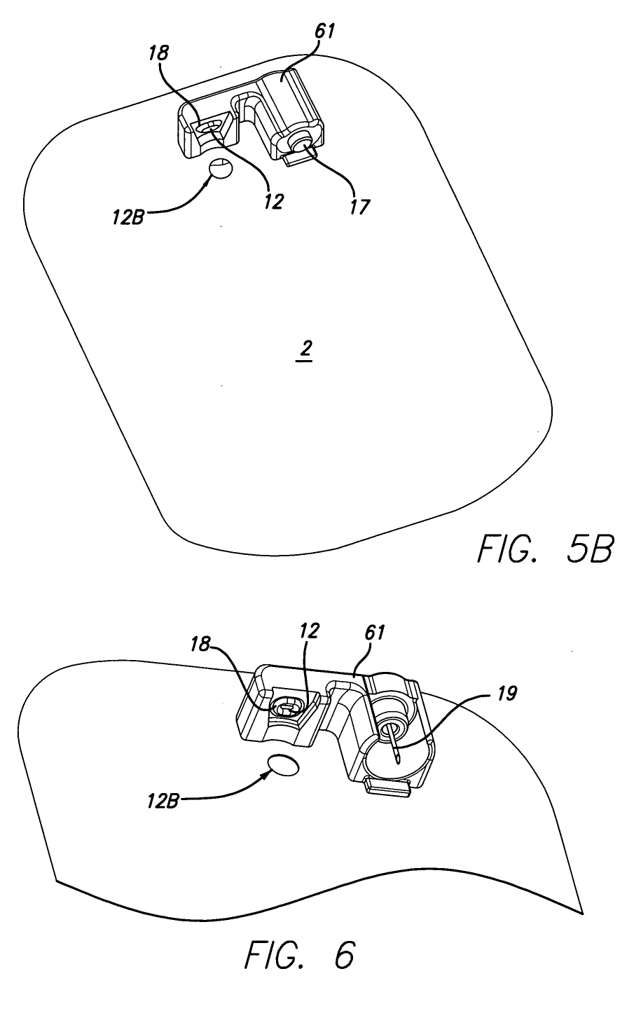

A connector part 3 is attached to or integrated with the surface plate 1. According to one embodiment the surface plate 1 and at least an outer cover of the connector part 3 is simply molded in one piece during manufacturing of the device. The connector part 3 forms a fluid path between e.g. a reservoir of medication or a reservoir for liquid collected from the patient and a cannula part 7. Therefore the connector part 3 is provided with at least two openings, one opening at each end of the fluid path (see FIGS. 5 and 6) where the first opening 13 is an inlet or outlet opening receiving or delivering fluid to a reservoir 6 and the second opening 12 is an inlet or outlet opening receiving or delivering fluid to a cannula part 7. The connection part 3 might be provided with extra openings e.g. for injection of a second medication or nutrient or for letting the fluid in the fluid path get in contact with a sensor. FIG. 1 shows the reservoir 6 attached to the connection part 3 at the first opening 13 of the connection part 3. In the following the first opening 13 will be referred to as “inlet” and the second opening 12 will be referred to as “outlet” although the direction of the flow through the fluid path is not significant for the invention.

The connection part 3 is further provided with a cannula opening 12A which accurately fits around a cannula part 7 when the cannula part 7 is mounted in the connection part 3 i.e. the cannula opening 12A has the same shape or profile as the cannula part 7 and is just big enough to let the cannula part 7 pass through and then fit into the opening. In FIG. 1 the cannula part 7 is shown in a position where the cannula part 7 is not fully inserted, normally the cannula part 7 would at this stage of insertion still be placed inside an inserter and it would not be visible. When the cannula part 7 is fully inserted, the upper surface i.e. the distal surface of the cannula part 7 is normally at level with or at a lower level than the outer surface of the connection part 3 around the cannula opening 12A.

When the cannula part 7 has been fully inserted into the connection part 3, an opening 20 in a side surface of the body 24 of the cannula part 7 corresponds to the opening 12 of the fluid path of the connection part 3 and fluid can flow from one part to the other. The opening 20 in the body 24 of the cannula part 7 might in the following be referred to as an “inlet” although the direction of the flow is not significant to the invention.

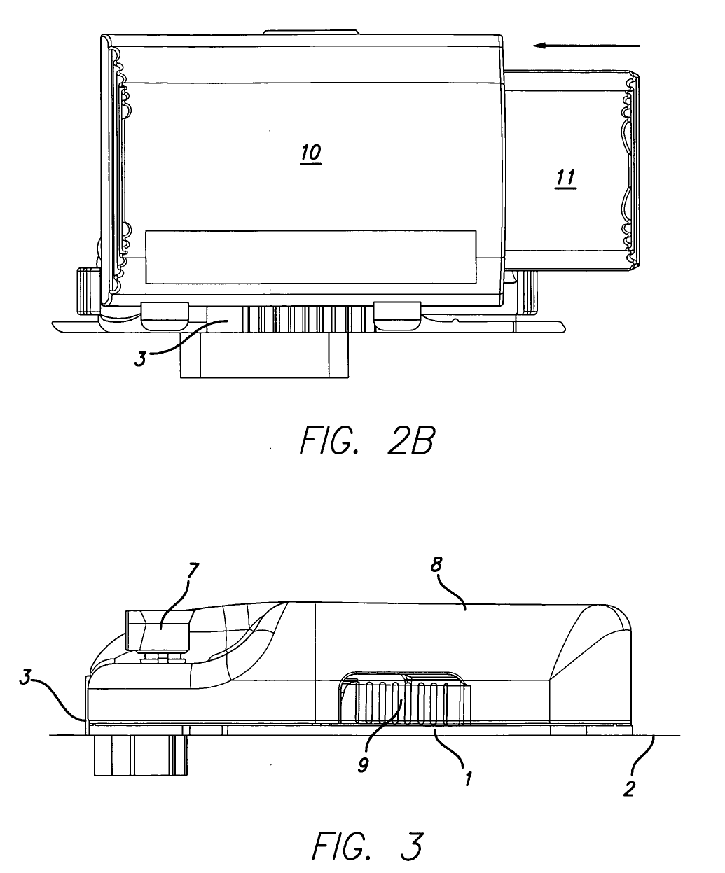

FIGS. 2A and 2B show a device which is ready to be placed on a patient's skin. FIG. 2A shows a side view of the inserter and FIG. 2B shows a view from the inserter-end of the device. The device comprises a base part according to the invention which base part comprises a surface plate 1 positioned unreleasably on a mounting pad 2 with an adhesive proximal surface. Releasably connected to the base part is a delivery part 8 and an inserter 10 having an actuator handle 11. The actuator handle 11 is in a pre-insertion position.

The delivery part 8 of this embodiment is joined to the base part by pushing the delivery part 8 down toward the guiding means 4 which in this case is a longitudinal raised platform having a metal lining 5 fastened to the top surface. The delivery part 8 is provided with a corresponding groove corresponding to the size of the metal lining 5 on the raised platform 4, in such a way that the corresponding groove of the delivery part 8 can slide along the metal lining 5 on the raised platform 4 of the base part in the longitudinal direction. When the delivery part 8 arrives at its working position, two release handles 9 engage respectively with two protruding parts 15 protruding from the upper surface of the surface plate 1. When the delivery part 8 is in its working position it is locked in any horizontal direction by the release handles 9 and the raised platform 4 with the metal lining 5 placed in the corresponding groove of the delivery part 8. These locking mechanisms make it possible to fasten and release the delivery device from the base part as often as needed i.e. a single-use base part can be combined with a multi-use delivery part.

The inserter 10 holds the cannula part 7 before insertion and the insertion is initiated by pushing a handle 11. FIG. 2B shows the direction the handle 11 has to be pushed in order to initiate insertion of the cannula part 7. After insertion a not shown insertion needle can be retracted to the inside of the inserter 10 and the inserter 10 can be removed from the base part, leaving an inserted cannula 22 fastened to the surface plate 1 of the base part. If the cannula 22 of the cannula part 7 is a hard self penetrating cannula there will be no separate insertion needle and therefore no need to retract the insertion needle.

FIG. 3 shows a side view of the same embodiment as shown in FIGS. 2A and 2B but in FIG. 3 the inserter has been removed. The cannula part 7 has the same position as it would have inside the inserter 10 before insertion.

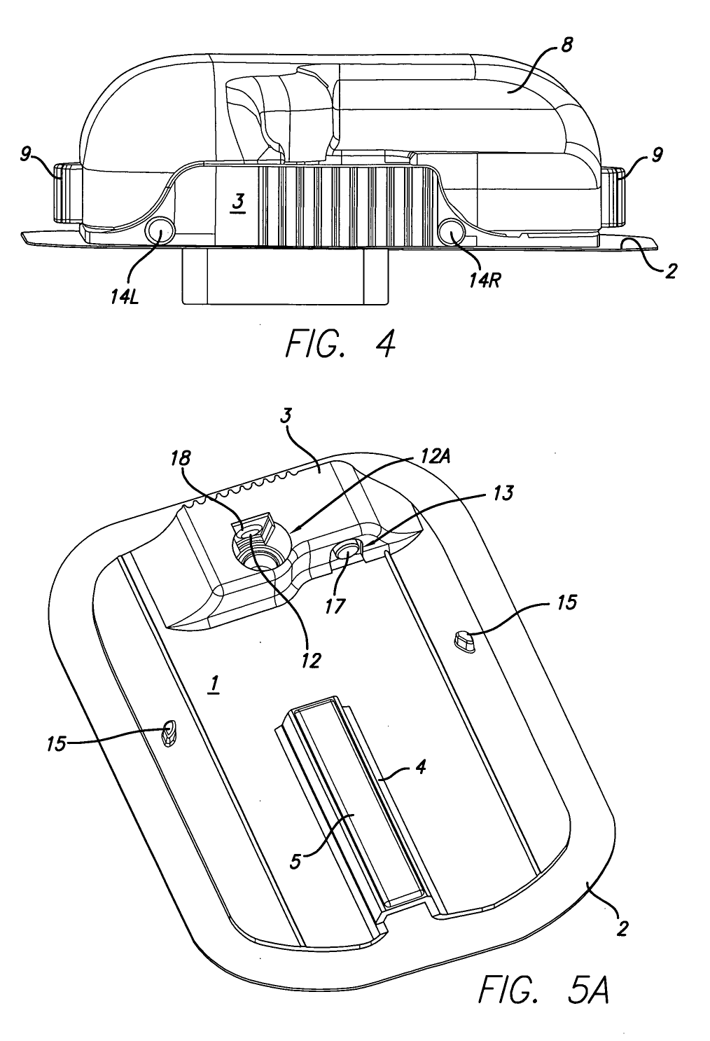

FIG. 4 shows the same embodiment as FIG. 3 but in FIG. 4 the assembly is shown from the end of the connection part 3 and the cannula part 7 has been removed. From this end it is possible to see the fastening means 14L and 14R of the base part which correspond to parts on the inserter 10. These fastening means comprise two openings 14L and 14R in the connection part 3 which correspond to two not shown protruding parts on the inserter 10. When the fastening means 14 on the base part is engaged with the corresponding fastening means on the inserter 10, the inserter 10 is prevented from moving in relation to the base part, at least in the direction perpendicular or a direction having a component perpendicular to the surface plate 1.

The FIGS. 5A and 5B show the connection part 3 of the base part. In FIG. 5A the connection part 3 is shown with an outer cover provided by the moulded surface plate 1 and in FIG. 5B the connection part 3 is shown without the outer cover i.e. only a mounting pad is shown. The outer cover shown in this embodiment is not a separate unit but is attached unreleasably to or simply made as a part of the surface plate 1 e.g. during a moulding process. The outer cover is provided with the cannula opening 12A for the cannula part 7 and an access opening 13 for the reservoir 6 thereby allowing access to the fluid path of the connection part 3 by the reservoir and the cannula part 7. The cannula opening 12A allows the cannula part 7 to be inserted sub- or transcutaneous into the patient within the circumference of the surface plate 1 and the contact surface 2 of the base part which in this embodiment is provided by a mounting pad is also provided with an opening 12B which allows for the cannula to be inserted (see FIG. 5B). This opening 12B is not necessary if the contact surface 2 is constructed of such a material and thickness that it can be penetrated by at least the cannula 22 of the cannula part 7.

In order to secure a fluid tight connection between the outlet opening 12 in the connection part 3 and the cannula part 7 the outlet opening 12 of the connection part 3 is provided with an elastic sealing 18 around the outlet opening 12. When the cannula part 7 is inserted it will be press fitted into the cannula opening 12A and the elastic sealing 18 will provide a completely fluid tight gasket around the corresponding openings 12 and 20. In order to improve the press-fitting and thereby the fluid tight connection between the cannula part 7 and the outlet of the fluid path, the cannula opening 12A can be provided with a decreasing cross-section in a plane parallel to the cannula 22 when inserted and perpendicular to the surface where the outlet of the fluid path is positioned. The cannula part 7 will have a corresponding decreasing cross-section.

In order to secure a fluid tight connection between the inlet opening 13 in the connection part 3 and the reservoir 6, a bubble shaped membrane 17 has been positioned in the first opening 13. The membrane 17 completely covers the inlet opening 13 (which according to this embodiment could be understood as being the open end of a connector needle 19) and prevents contamination of the connection part 3. When the reservoir 6 is pressed towards the connection part 3, the connector needle 19 will penetrate the membrane 17 and provide a completely fluid tight transferal of fluid between the connection part 3 and the reservoir 6.

That the membrane 17 is bubble shaped means that it is attached around the opening in a way where it surrounds the opening—often it is placed at the edge of the opening it protects—and the membrane 17 protrudes from the planed formed by the edge of the opening and forms a dome in a distance from the edge. The height or length of the dome can correspond to the length of a connector needle 19.

In FIG. 6 the connector needle 19 is shown as being a part of the connection part 3 i.e. it is attached to the connection part 3 but the connector needle 19 might just as well be a part of the reservoir 6.

According to one embodiment the connection part 3 is provided with both a connector needle 19 and a bubble shaped self closing membrane 17 and the reservoir 6 is also provided with a bubble shaped self closing membrane. As both parts are provided with self closing membranes it will be possible to separate the two units from each other and rejoin them at a later time without the connection part 3 and thereby the patient being contaminated.

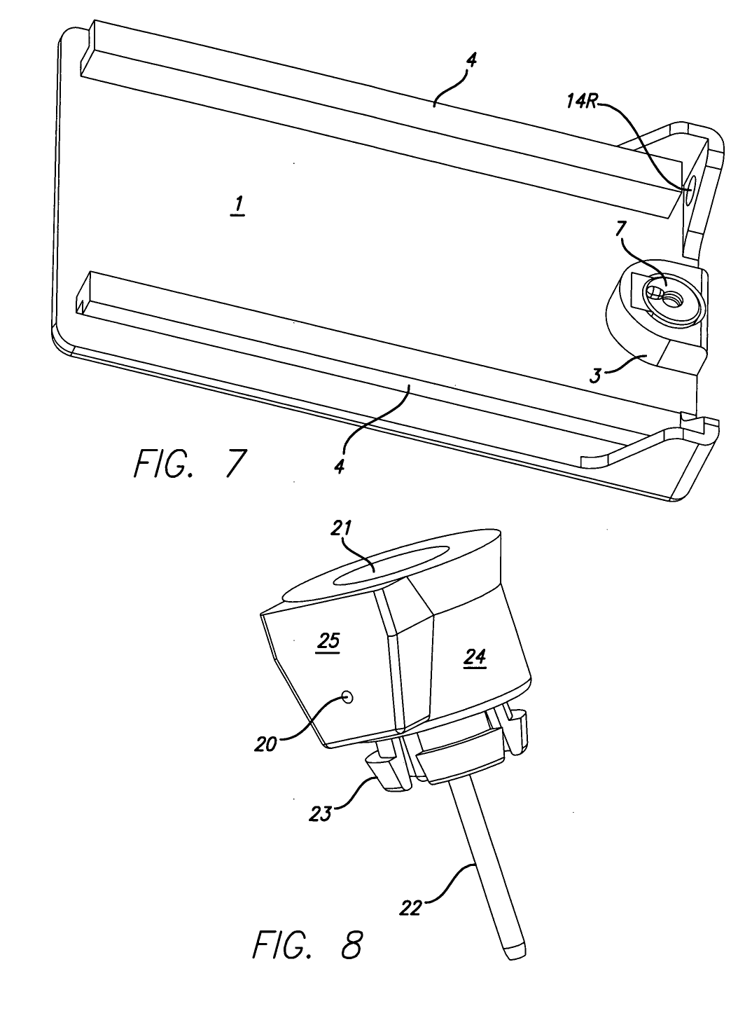

FIG. 7 shows a second embodiment of the base part. This embodiment is provided with two guiding means 4 in the form of two right angled profiles shaped as: ┐ ┌, and protruding from the surface plate 1 of the base part. The guiding means 4 correspond to means on a delivery part or a cover which is to be fastened to the base part. Such corresponding means can e.g. be formed as one or more hooks having a profile in the form of ┘ and └.

The fluid path of the connection part 3 is very short compared to the embodiment shown in FIG. 1-6 and the inlet of the connection part 3 is placed in a centre position in relation to the guiding means 4 but the inserted cannula part 7 has the same profile as the cannula part 7 fitted to the embodiment of FIG. 1-6.

FIG. 8 shows an enlargement of the cannula part 7 shown in FIG. 1. This embodiment comprises a body 24 provided a cannula 22 and with a protruding front 25 having a flat surface. The surface of the cannula part 7 having an opening need not be flat; it can actually have any desired shape as long as it is possible to create a corresponding surface on the connection part 3 facing the cannula part 7. In one embodiment the front 25 is inclined in such a way that the cross-section at the upper i.e. distal end is larger than the cross-section at the proximal end, i.e. the end closest to the patient after insertion, of the front. The front 25 is provided with an opening 20 through which liquid can exit or enter the cannula part 7. The body 24 is further provided with a top opening 21 which opening can be covered with a self closing membrane. The opening 21 need some kind of entrance protection as it is facing an outer surface which is in contact with the surroundings. The top opening 21 is primarily used when inserting the cannula part 7 if the cannula 22 is a soft cannula. That the cannula 22 is soft means that is made of a relatively soft material which can not penetrate the patients skin, in this case it is necessary to use a pointy insertion needle of a relatively hard material when inserting the cannula and this pointy needle can be inserted through the top opening 21, pass through an inner hollow in the body 24 of the cannula part and further pass through the full length of the cannula 22 in such a way that the pointy end of the insertion needle stick out of the open end of the hollow cannula 22. After insertion i.e. after the cannula 22 has been placed sub- or transcutaneous in the patient, then the insertion needle is retracted and the cannula 22 is left inside the patient.

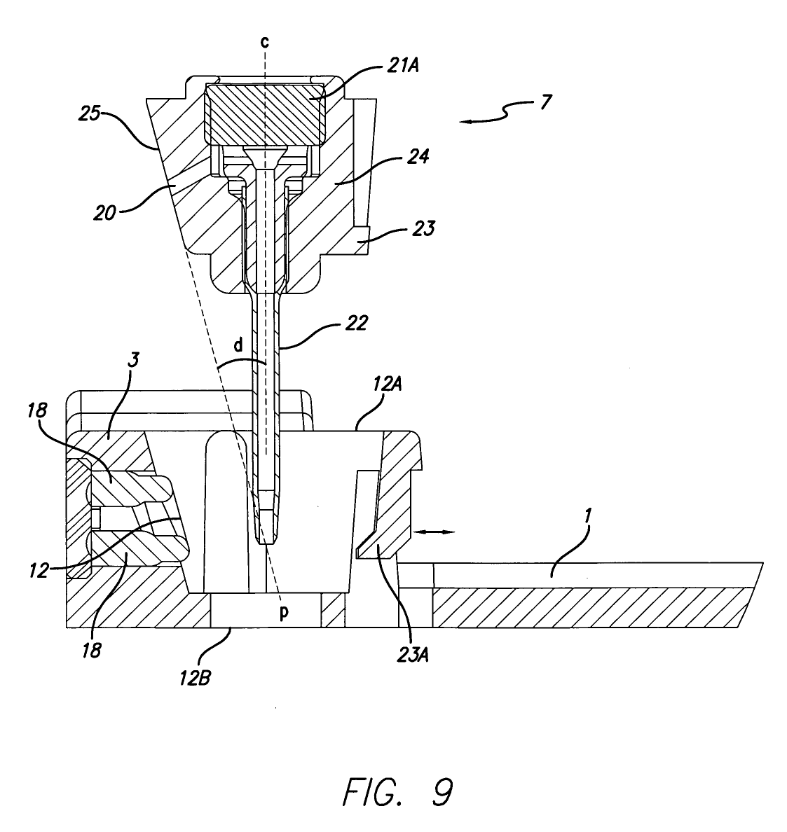

FIG. 9 shows an enlargement of a second embodiment a cannula part 7. This embodiment also comprises a body 24 provided with a cannula 22 and with a protruding front 25 having a flat surface provided with an opening 20 but according to this embodiment the protruding front 25 is inclined in such a way that the pressure between the opening 20 and the sealing 18 around the second opening 12 of the connection part 3 is increased. The inclination of the front 25 is defined by the angle d between the centre line c of the cannula 22 and a line parallel to the surface around the opening 20. The angle d will be larger than 0° and smaller than 90°, normally d ∈ ]0°, 30°] depending on the diameter or the protrusion of the sealing 18 or [60°, 90°[. The distance d₁ between at the distal end of the surface of the protruding part 25, i.e. the end of the cannula part 7 which is furthest away from the patient after insertion, and the centre c of the cannula part 7 is larger than the distance d₂ between the surface of the protruding part 25 at the proximal end, i.e. the end closest to the patient after insertion, and the centre c of the cannula part 7. Normally the distance d₂ will be so small that the proximal end of the protruding front 25 does not touch the sealing 18 of the connection part 3 during insertion.

In one embodiment (not shown) the angle d is close to about 90° i.e. d=90°, such an embodiment would in a drawing corresponding to FIGS. 9A and 9B appear to have an upward opening 12 of the connection part 3 fitting to a downward opening 20 of the cannula part 7. This means that the force pushing the cannula part 7 toward the sealing 18 will be close to perpendicular to the contact surface of the sealing 18 and this will prevent that the sealing is distorted during insertion of the cannula part 7 by the cannula part 7 sliding along the sealing 18.

In another embodiment (shown in FIG. 8) d is about 0° as the protruding front 25 and the centre line c are parallel. According to this embodiment the cannula part 7 will be in sliding contact with the protruding sealing 18 which can cause the sealing to be distorted.

As according to the embodiment of FIG. 8 the protruding front 25 of the cannula part 7 need not be flat; it can actually have any desired shape as long as it is possible to create a corresponding surface on the connection part 3 facing the cannula part 7. Also the opening 20 of the protruding front 25 can be an inlet or an outlet depending on the purpose of the cannula part 7. In FIG. 9 which is a cut-through view it is shown how the top opening 21 of the body 24 is covered with a self closing membrane 21A. As according to the embodiment of FIG. 8 the top opening 21 is primarily used when inserting the cannula part 7 if the cannula 22 is a soft cannula but the top opening 21 can also be used to inject medication or nutrients other than the primary medication which could be e.g. insulin which the patient receive via the opening 20.

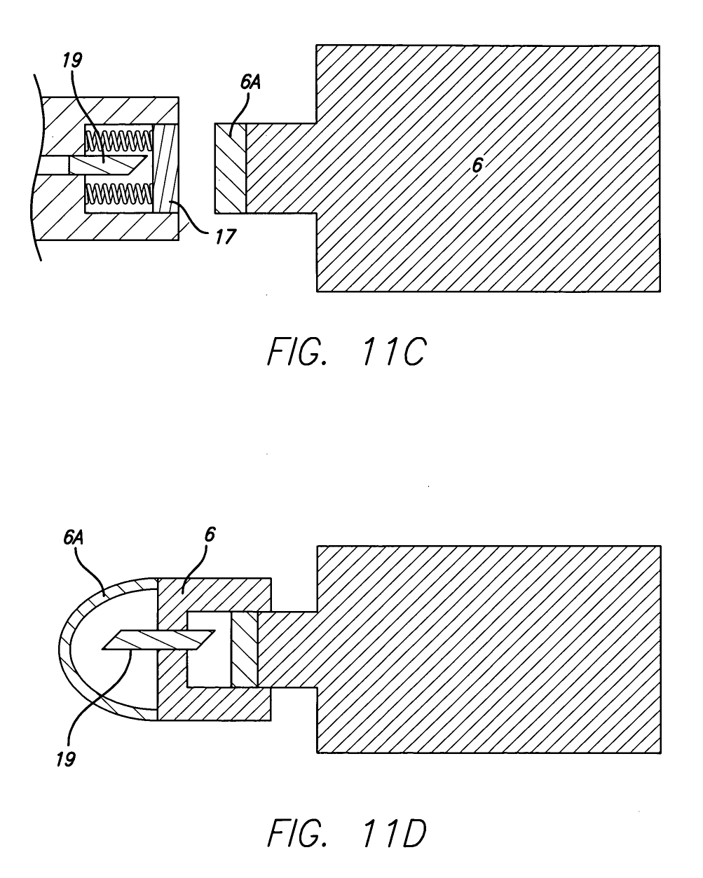

This embodiment of the cannula part 7 is also provided with fastening means 23 and in this embodiment the fastening means 23 has the form of a protruding part 23 on the cannula part 7 which corresponds to a flexible part 23A on the stationary base part. The flexible part 23A can be pushed outward as indicated with an arrow at FIG. 11 when the protruding part 23 on the cannula part 7 passes during insertion of the cannula part 7. After insertion the upward surface of the protruding part 23 of the cannula part 7 will be locked by the downward surface of the flexible part 23A of the base part and it will not be possible to detach the cannula part 7 from the base part.

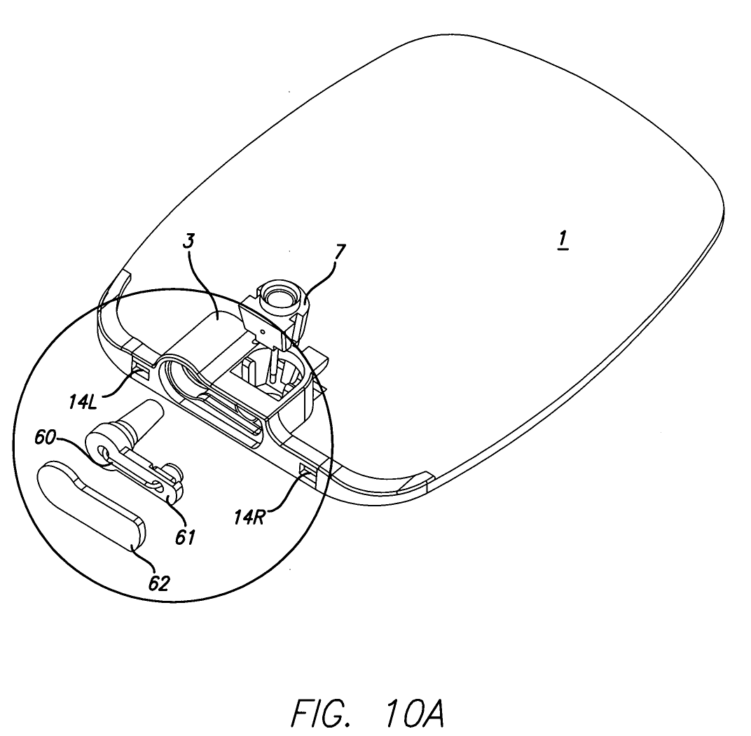

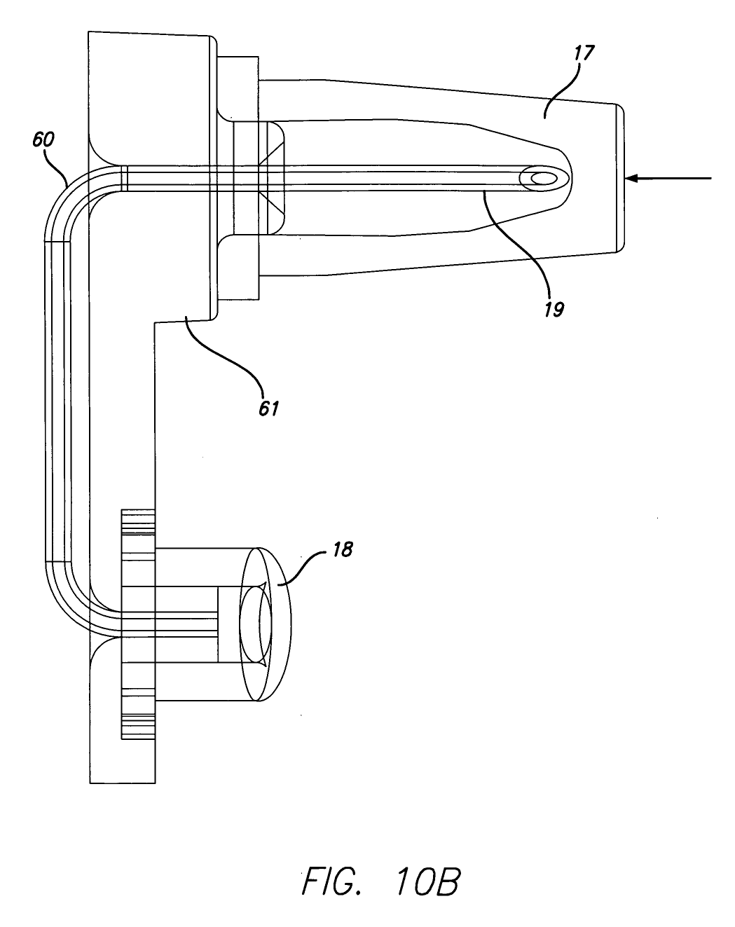

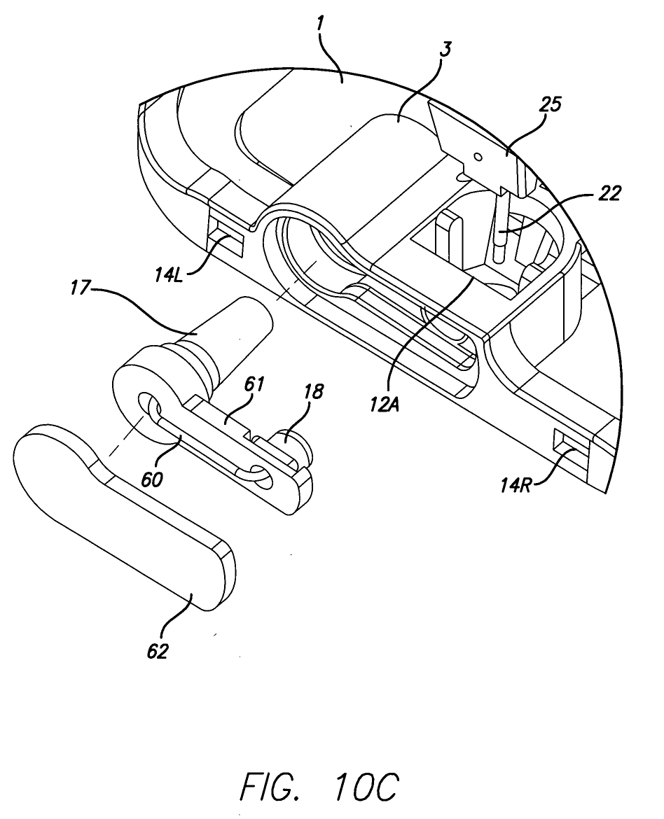

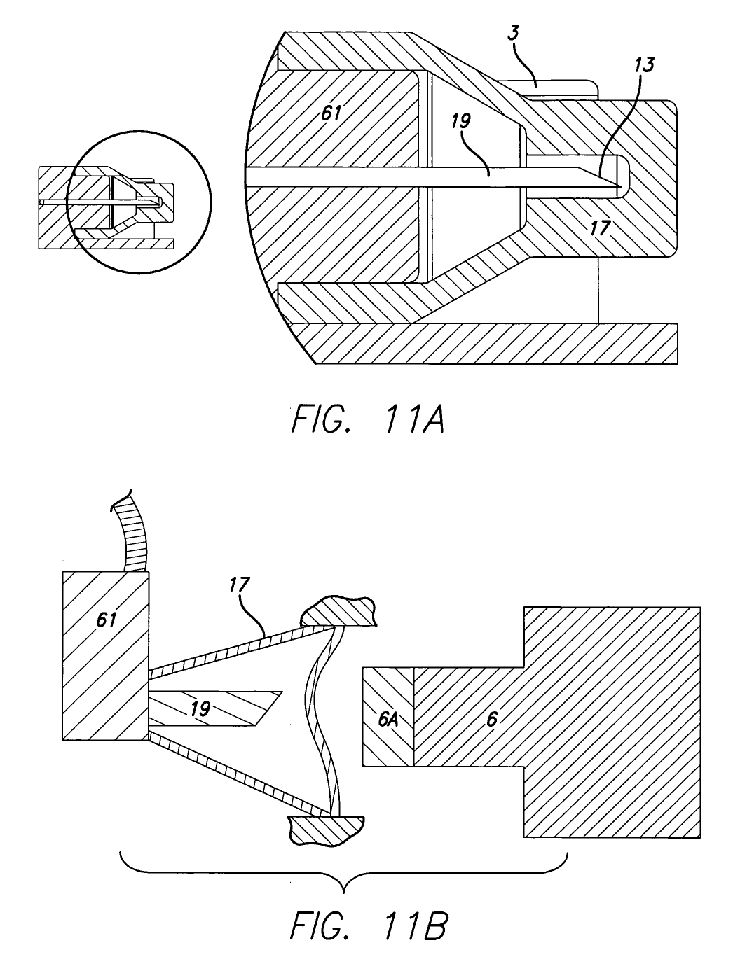

FIGS. 10A-C show one embodiment of a connection part 3. FIG. 10A show the embodiment of the connection part 3 in an exploded view where the internal holding parts 61 for a tube 60 providing a fluid path is shown. FIG. 10B shows a cut through the internal holding part 61 according to which it is possible to the position of the tube 60. FIG. 10C shows an enlargement of the encircled part of FIG. 10A.

According to the present embodiment the connection part 3 and the surface plate 1 is molded in one piece of a plastic material, the connection part is provided with several openings, one opening 12A is prepared for fitting in the cannula part 7 and another opening is prepared for fitting in the internal parts of the connection part 3. The internal parts of the connection part 3 according to this embodiment comprises one tube which at two positions are bend in 90° i.e. both the inlet and the outlet end of the tube 60 points in the same direction perpendicular to the connecting part of the tube 60 where the connecting part of the tube 60 forms the fluid path between the two bending parts.

At one end the tube 60 is protected by a bubble shaped membrane 17 and at the other end the tube 60 is open and unprotected, but the open tube end is surrounded by a sealing 18 which is attached unreleasably to a holding part 61. When the internal parts has been placed in the corresponding opening in the connection part 3 a cover 62 accurately fitting in the opening is placed in level with the surface of the connection part 3 in such a way that the user experience a smooth surface which cannot be tampered with.

FIG. 10B shows an enlargement of the internal parts of the connection part 3. The holding parts 61 comprise a single molded part which is providing a stable embedment of the tube 60. The open end of the tube 60 opens into a space surrounded by the sealing 18. The closed end of the tube 60 is completely surrounded by a soft membrane. “Completely surrounded” means that the there is no free access to the surroundings, “soft membrane” means that the membrane can be penetrated by a needle, especially the connector needle 19 which is provided by the end of the tube 60 and which is embedded inside the soft membrane. The end of the tube 60 which constitutes the connector needle 19 is in this embodiment not actually in touch with the surrounding membrane 17. The connector needle 19 is surrounded by air, and the internal space surrounding the connector needle 19 has a cylindrical or conical shape i.e. a circular cross-section. The walls of the membrane 17 will deform by bending inwards or outwards when the length of the membrane is reduced as a result of the applied pressure.

FIG. 10C shows an enlargement of the enclosed field marked in FIG. 10A.

FIG. 11A shows an enlargement of an embodiment of a bubble membrane 7. This bubble membrane 17 completely surrounds the part of the connector needle 19 which protrudes from the surface of the holding part 61 in which the connector needle 19 is embedded. The connector needle 19 does not touch the bubble membrane 17 when no pressure is put on the membrane 17 i.e. the connector needle 19 is completely surrounded by air which makes it possible to gas sterilize the connector needle 19; this is the state in which the membrane is shown in the figure. The tip of the connector needle is surrounded of membrane parts with quite thick walls, while the part of the membrane closest to the holding part has walls of approximately half this thickness, this has the result that when pressure is put on the membrane the thick walled part does not change shape, in stead the part of the membrane having reduced wall thickness i.e. the part closest to the holding part will give in and be pressed toward the holding part 61.

FIG. 11B shows another embodiment of a bubble shaped membrane 17. According to this embodiment the reservoir 6 which is provided with an entrance protecting membrane 6A is pushed toward the membrane covered connector needle 19. The bubble membrane 17 is made of a flexible material which makes it possible for the membrane to be deformed to such an extent that the connector needle 19 can penetrate the protecting membrane 6A and assure access to the fluid reservoir 6.

FIG. 11C shows yet another embodiment of a membrane 17 protecting the opening to the connection part 3. This membrane 17 is not bubble shaped but it provides a wall in a space surrounding the connector needle 19. The wall is pliant i.e. it will move back when the reservoir is pressed against it. The membrane wall 17 is kept in position by one or more springs i.e. the membrane 17 is able to return to the original position when the pressure from the reservoir 6 which keeps it in place is released. The opening in which the membrane slides back and forth closely fits the connecting part of the reservoir 6.

FIG. 11D shows another embodiment of a reservoir 6 where a bubble membrane 6A is mounted at the outlet of the reservoir 6 which outlet is connected to the fluid path of the connection path 3. The not shown end of the fluid path connecting to the reservoir 6 is provided with a membrane protecting the entrance of the fluid path during periods where the fluid path is not connected to the reservoir 6. According to this embodiment the fluid path need not be provided with a connector needle 19 as the connector needle 19 according to this embodiment is part of the reservoir 6.

FIG. 11E shows yet another embodiment of a bubble membrane 17 and how the reservoir is pressed against the connector needle 19 in order to provide a fluid path for the medication contained in the reservoir 6. The bubble membrane 17 is flexible and is able to be reduced in size in such a way that it allows the entrance of the reservoir 6 to be pressed into the opening in the connection part 3 which surrounds the membrane 17 and the connector needle 19 i.e. the length of the membrane 17 can be reduced without the diameter of the membrane 17 need to be extended. According to the shown embodiment the material of the membrane will be folded inwards.

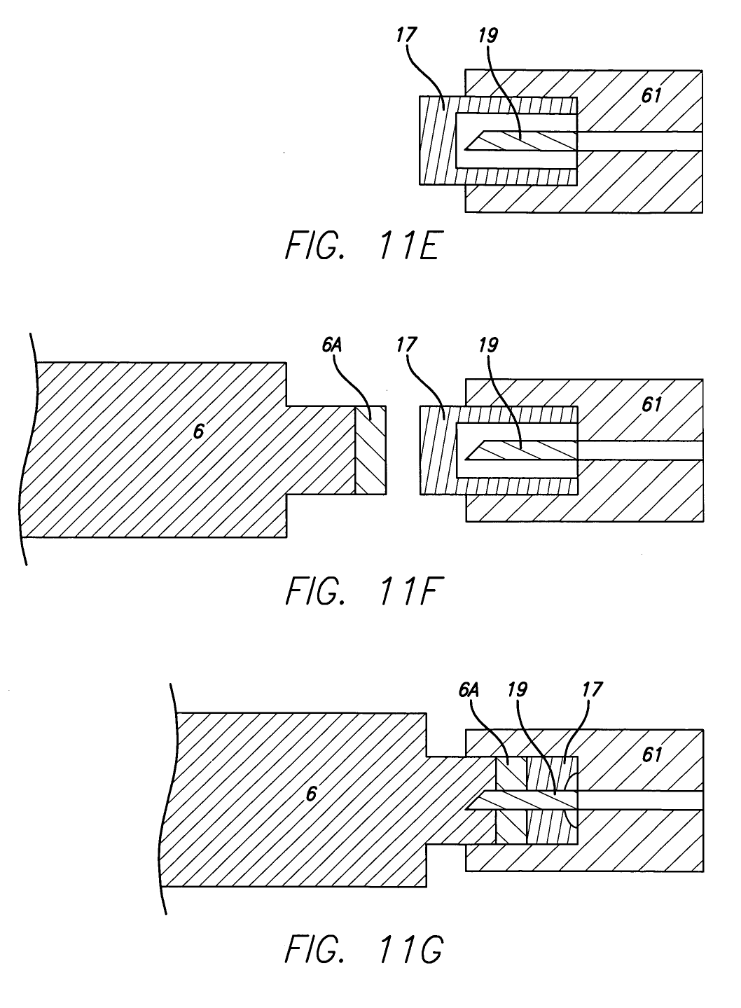

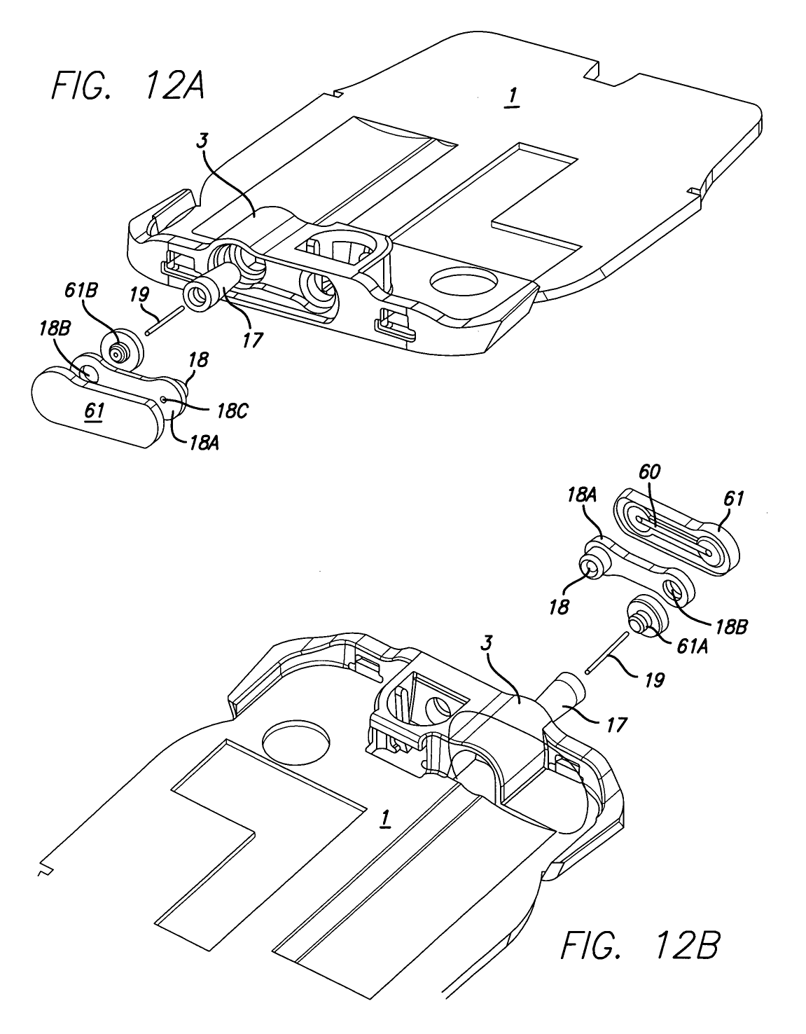

FIGS. 12A and 12B show an alternative embodiment which provides a fluid tight connection when transferring liquid from the reservoir 6 to the cannula part 7. Both in FIGS. 12A and 12B the internal parts of the connection part 3 are presented in exploded form.

The internal parts of the connection part 3 according to this embodiment comprises a holding part 61 for a tube 60, fastening means 18a for a sealing around the second opening 12, fastening means 61a for the soft bubble membrane 17 separated from the holding part 61 and provided with a sealing opposite of the fastening means 61a, a connector needle 19 and a soft bubble membrane 17.

The tube 60 can be constituted either by a straight or by a bent piece of pipe-shaped rigid material such as steel or a hard plastic material, and the tube 60 is held by a holding part 61 which holding part 61 according to this embodiment also provides for a cover surface which cover surface is constituted by the outer side of the holding part 61 shown in FIG. 12A. If the tube 60 is a straight piece of material the second end of the tube 60 which is the end connected to the cannula part 7, opens into a room formed in the holding part 61 and having an inlet/outlet to the cannula part 7 through a small opening 18c in the fastening means for the sealing 18a. If the tube 60 is a bent piece of material the second end of the tube 60 is bent in about a 90 degree angle through the small opening 18c which is formed in the fastening means 18a. The fastening means 18a provide the means for fastening of the sealing 18 to the holding part 61. The fastening means 18a are provided with a large opening 18b which opening provides a connecting room between the tube 60 and the connector needle 19 when the holding part 61, the fastening means 18a and a separate sealing part 61 b are pressed together. The connecting room provides a fluid connection between the connector needle 19 and the tube 60 as the room can fill with fluid entering from the connector needle 19 where after fluid can exit the connecting room by exciting through the tube 60. The side of the fastening means 61a for the membrane which is turned toward the fastening means 18a for the sealing can be provided with a sealing in order to keep the connecting room fluid tight.

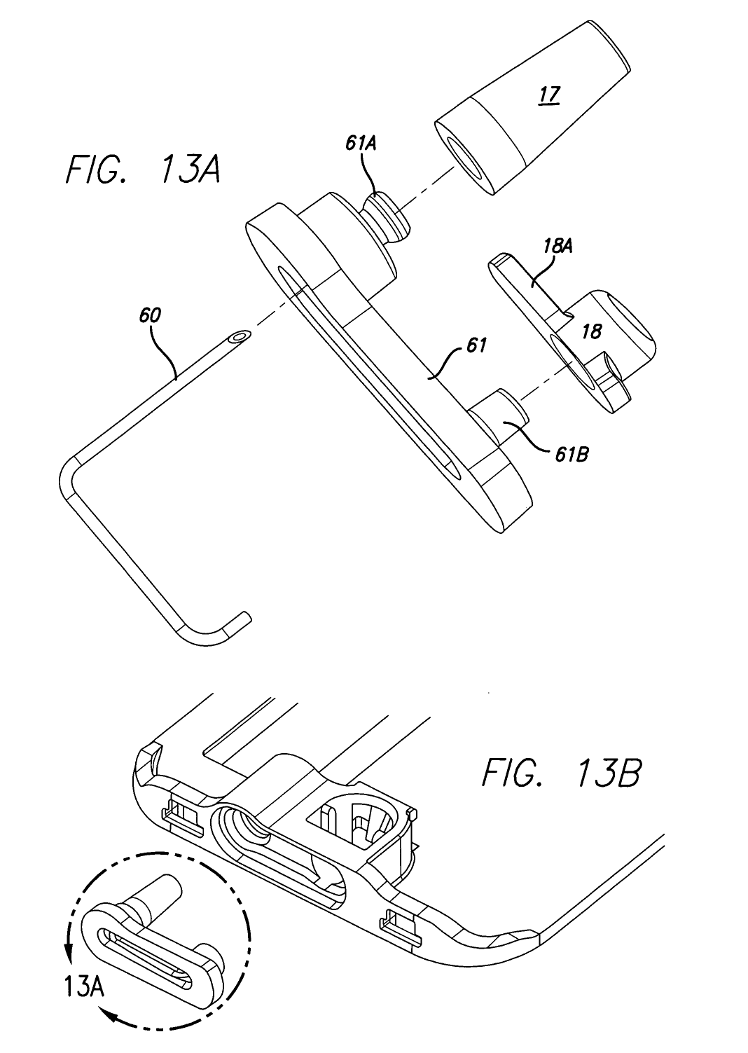

FIGS. 13A and 13B show another embodiment of the connection part compared to the connection part shown in FIG. 10A-C, which connection part 3 comprises the same units. FIG. 13B shows where internal parts of the connection part 3 are positioned in relation to the outer parts of the connection part 3. FIG. 13A shows an exploded view of the internal parts of the connection part 3 which internal parts are encircled in FIG. 13B.

Like in FIG. 10A-C the holding parts 61 comprise a single molded part. The holding part 61 provides a stable embedment of the tube 60, the holding part 61 is normally molded in one part but it might be formed by joining two or more smaller parts. Such smaller parts could be joined by welding or gluing. As the holding part 61 is rather small, normally less than 2 cm in length, it can be difficult to join the smaller parts.

The tube 60 has two open ends, i.e. liquid can pass in or out, and when the tube 60 is mounted in the holding parts 61, the first open end opens into a space surrounded by the closed soft membrane 17 and the second open end opens into a space surrounded by the sealing 18.

The first end of the tube 60 is pointed i.e. sharp and can provide a connection to the reservoir 6 as this first end of the tube 60 can penetrate both the closed soft membrane 17 surrounding the open end of the tube 60 and a membrane 6A protecting the inlet to the reservoir 6. Like in the embodiment shown in FIG. 10A-C this end of the tube 60 is completely surrounded by a soft membrane 17 where “completely surrounded” means that there is no free access from the first open end of the tube 60 to the surroundings, “soft membrane” means that the membrane can be penetrated by a needle, especially the connector needle 19 provided by the end of the tube 60. The end of the tube 60 which constitutes the connector needle 19 is in this embodiment not in touch with the surrounding membrane 17 when the soft membrane 17 is not influenced by impacts from the surroundings. The soft membrane 17 is according to this embodiment fastened to the holding part 61 by pressing the relatively soft and compliant membrane material against the holding part 61, the edge of the membrane 17 being closest to the holding part 61 can expand in diameter and slide over a mushroom shaped fastening part 61a which is an unreleasable part of the holding parts 61. When the soft membrane 17 is in its final position, the extended diameter of the membrane 17 can return to a smaller size and this reduction of the diameter will keep the membrane 17 in place around the fastening part 61a. The fastening of the membrane 17 is enhanced if the membrane 17 is provided with one or more inward protruding parts which will rest against the part of the fastening means 61a being closest to the holding parts 61 and having the smallest diameter after mounting of the membrane 17.

The second open end of the tube 60 is blunt and opens into a closed ring of sealing 18 i.e. the sealing has the form of a short pipe and do not stop the flow of liquid in or out of the tube 60. The sealing 18 is fastened to the holding parts 61 by fastening means 18a, the fastening means 18a makes it easier to e.g. weld or glue the sealing 18 unreleasably to the holding part 61.

The tube 60 is formed in one piece; normally it will be made of steel or a hard plastic material. If the tube is formed with a pointed end which is to penetrate the soft membrane 17 during use, it should at least be made of a material which is hard enough to penetrate the soft membrane 17 and e.g. the membrane 6A covering the inlet to the reservoir 6. It is possible to construct the tube 60 with two blunt ends, according to such an embodiment the reservoir 6 could be provided with a connector needle 19 which could penetrate the soft membrane 17 when transferring liquid to the cannula part 7.

According to the embodiment of FIG. 13A-B, the tube 60 is bent at two positions. This is suitable according to this embodiment as the reservoir 6 and the cannula part 7 are mounted on the same side relative to the holding parts 61. The angles of both the bends are about 90 degrees, if the tube 60 is to be positioned in a one piece holding parts 61 by pushing, then the two legs provided by these two bends should have the same angle in relation to the connecting tube piece between the two bends but the angles need not be 90 degrees. If the reservoir 6 and the cannula part 7 are positioned different in relation to each other the tube 60 might be bend only once e.g. in the situation where the cannula part 7 is positioned close to the edge of the surface plate 1 and has the front provided with the opening 20 turned toward the first and only bending of the tube 60.

According to one embodiment the tube 60 comprises a hollow needle e.g. made of steel. Such a needle can easily be manufactured at an automated process at a low price. Also such a needle can easily be bending in one or more positions in order to satisfy any need there would be for positioning of the needle between the reservoir 6 and the cannula part 7. Whether the needle is provided with blunt or pointed ends can depend on the parts corresponding at the ends of the needle but normally the needle will be provided with at least one pointed or sharp end which is able to penetrate a protective membrane.

Also if the connection part 3 is placed on a middle or central part of the surface plate 1, then the reservoir 6 could be placed at one side of the connection part 3 at the first end of the tube 60 and the cannula part 7 could be placed at the opposite side of the connection part 3 at the second end of the tube 60 and then the tube 60 could be straight without any bending.

According to the present invention the tube 60 is stationary relative to the surface plate 1 after the tube 60 has been positioned in the holding part 61 and mounted on the surface plate 1. That the tube 60 is stationary means that it does not pivot or in any way move back or forth in relation to the surface plate 1, the tube 60 simply serves as a path for transporting liquid.

| Ref No | Name |
|---|---|
| 1 | Surface plate |
| 2 | Mounting pad |
| 3 | Connection part |
| 4 | Guiding means |
| 5 | Metal lining |
| 6 | Reservoir |
| 6A | Membrane for reservoir |
| 7 | Cannula part |
| 8 | Delivery part |
| 9 | Release handles of delivery part |
| 10 | Inserter |
| 11 | Actuator handle for inserter |
| 12 | Outlet or Second opening |
| 12A | Cannula opening |
| 12B | Opening in surface plate for cannula part |
| 13 | Inlet or First opening |
| 14 | Fastening means for inserter |
| 15 | Protruding parts of base part |
| 16 | — |
| 17 | Bubble membrane |
| 18 | Sealing around outlet or second opening |
| 18a | Fastening means for sealing |
| 18b | Large opening in fastening means 18a |
| 18c | Small opening in fastening means 18b |
| 19 | Connector needle |
| 20 | Opening into cannula part |
| 21 | Top opening in cannula part |
| 22 | Cannula |
| 23 | Fastening means |
| 24 | Body of cannula part |
| 25 | Protruding front |
| 60 | Tube |
| 61 | Holding part for tube |
| 61a | Fastening means for membrane |
| 61b | Separate sealing part |
| 62 | Cover |
| 63 | Molded fluid path |

1. A fluid connection for use between a reservoir and a cannula part of a medical device, the fluid connection comprising:
a tube of rigid material, the tube having a first end operably connected to the reservoir and a second end operably connected to the cannula part, the tube being operably attached to a surface plate of the medical device.

2. A fluid connection according to claim 1, wherein the fluid connection is fastened to the surface plate by a holding part.

3. A fluid connection according to claim 1, wherein the tube comprises metal or plastic.

4. A fluid connection according to claim 3, wherein the tube comprises a hollow needle.

5. A fluid connection according to claim 1, wherein the tube has a diameter or maximum cross-section less than or equal to about 1 mm.

6. A fluid connection according to claim 1, wherein the tube has at least one pointed end protruding from a holding part.

7. A fluid connection according to claim 6, wherein the tube has a blunt end.

8. A fluid connection according to claim 6, wherein the pointed end of the tube forms a connector needle forming an inlet to a connector part and when pushing a reservoir towards the inlet, the connector needle penetrates a membrane completely covering a first opening of the connector part.

9. A fluid connection according to any of the claims 1, wherein the tube comprises a single piece.

10. A fluid connection according to claim 9, wherein the tube is bent at an angle greater than 0 degrees in at least one position.

11. A fluid connection according to claim 10, wherein the tube is bent in an angle greater than 0 degrees in at least two positions.

12. A fluid connection according to claim 11, wherein the angles of the two positions are identical.

13. A fluid connection according to claim 1, wherein the tube is stationary in relation to the surface plate after the tube has been attached to the surface plate.

14. A fluid connection according to claim 1, wherein the length of a fluid path provided by the connection is maximally about 3 cm.

15. A fluid connection for use between a reservoir and a cannula part of a medical device, the fluid connection comprising:
a tube of rigid material, the tube having a first end operably connected to the reservoir and a second end operably connected to the cannula part;
a holding part configured to secure at least a portion of the tube therein;
a membrane surrounding the first end of the tube to protect the first end; and
a sealing portion connected to the holding part and surrounding the second end.

16. The fluid connection according to claim 15, wherein the tube is bent at an angle greater than 0 degrees in at least one position.

17. The fluid connection according to claim 15, wherein the medical device further comprises a surface plate and the fluid connection is operably connected to the surface plate.

18. The fluid connection according to claim 15, wherein the first end is pointed to form a connector needle.

19. The fluid connection according to claim 15, wherein the holding part comprises a single molded part.

20. The fluid connection according to claim 15, further comprising a cover for covering the holding part with the tube secured in the holding part.
